(19) United States
(12) Patent Application Publication
(10) Pub. No.: US 2022/0105228 A1
(43) Pub. Date: Apr. 7, 2022

(54) System and Method for Disinfecting Indoor Environments

(71) Applicant: Pure Lighting Company, Kinnelon, NJ (US)

(72) Inventor: Michael Litvin, Kinnelon, NJ (US)

(73) Assignee: Pure Lighting Company, Kinnelon, NJ (US)

(21) Appl. No.: 17/494,126

(22) Filed: Oct. 5, 2021

Related U.S. Application Data
(60) Provisional application No. 63/087,379, filed on Oct. 5, 2020.

(51) Int. Cl. A61L 9/20; G01P 13/00

(52) U.S. Cl. CPC A61L 9/20; A61L 2209/12; A61L 2209/111; G01P 13/00

(57) ABSTRACT

A system and method for disinfecting an indoor environment. The system includes a user computer having a user app operably coupled with a controller. The user computer communicates with the controller through a network. A UV-C lamp and motion sensor are operably coupled with the controller. The UV-C lamp is operable in a first mode when the indoor environment is occupied to emit ultraviolet radiation to an upper section of the indoor environment at a first level, and a second mode when the indoor environment is unoccupied to emit ultraviolet radiation to sections of the indoor environment beyond the upper section at a level greater than the first level. The method includes the steps of programming parameters for controlling the UV-C lamp remotely, communicating to and storing the parameters on the controller, and controlling the UV-C lamp for operation in the first and second modes.

### CROSS-REFERENCE TO RELATED APPLICATION

This application claims priority to U.S. Provisional App. No. 63/087,379, titled “Disinfection System and Method,” filed on Oct. 5, 2020, which is incorporated by reference in its entirety.

### FIELD

The present invention relates to disinfection systems and methods, and in particular to a system and method disinfecting indoor environments using ultraviolet radiation.

### BACKGROUND

Airborne infections spread when bacteria or viruses travel on dust particles or small respiratory droplets that become aerosolized when an infected person sneezes or coughs. Healthy people can inhale the infectious droplets, or the droplets can land on their eyes, nose and mouth.

One such airborne infection is COVID-19, which has caused many illnesses and deaths, and has affected daily activities of many. People are especially vigilant about spending time indoors because often times indoor air is not recycled with fresh outdoor air, and because many indoor environments have poor circulation. Thus, a need exists for disinfecting indoor environments to ensure that individuals are safe from contracting airborne infections.

One method of disinfecting indoor environments is by utilizing ultraviolet radiation (UVR), more specifically, UV-C, which is UVR with wavelengths between 100 and 280 nm. Currently, there are two distinct types concerning UV-C disinfection: Upper Room UV-C and Whole Room UV-C (UV-C Air and Surface). Such existing systems include, for example, Puro Lighting—Helo F1, Healthe Lighting—Cleanse Retrofit Troffer, Cooper Lighting Solutions—GSL Germicidal UV Striplight and American Ultraviolet—TB, RAM Series.

Upper Room UV-C systems treat the room during periods of occupancy but are limited to a low threshold limit value. As such, even though Upper Room UV-C systems can function in occupied spaces, their efficacy in deactivating pathogens is limited by an output threshold limit value not to exceed 6.0 mJ/cm2 at 254 nm by the American Conference of Governmental Industrial Hygienists (ACGIH) Committee on Physical Agents. This threshold limits the efficacy of Upper Room UV-C systems with research showing effectiveness not exceeding 80%, as shown at https://stacks.cdc.gov/view/cdc/11285.

Whole-Room UV-C systems operate at much higher output. Whole Room UV-C disinfection systems have shown 99.9% or higher inactivation rates of pathogens, as shown at https://www.ncbi.nlm.nih.gov/pmc/articles/PMC5369231/. However, Whole Room UV-C disinfection systems should only be activated during periods of non-occupancy as excessive exposure can lead to corneal irritation and skin reddening and irritation (photodermatitis).

The dose delivered by a particular product is based on the UV-C irradiance and the duration of exposure. As a result, 4-log reductions of aerosolized viruses, bacteria, and fungi were achieved at dosages of 25 mJ/cm2 or less in a Whole Room UV-C treatment test, as shown at https://aem.asm.org/content/84/17/e00944-18.

Therefore, a need exists for a hybrid Upper Room and Whole Room UV-C system and method that maximizes the air and surface disinfection benefits of both existing systems for operation during occupancy and non-occupancy.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

The purpose of the present invention is to provide an automated and comprehensive disinfection of pathogens in facilities during periods of occupancy and non-occupancy. The advantage of a hybrid UV-C system and method of the present invention that emits short-term highly effective treatments via Whole-Room operation and long-term treatments during periods of occupancy via Upper Room operation is very clear. The system and method of the present invention will not only provide high effective UV-C dosage treatments during periods of occupancy and vacancy, but of even more importance, the hybrid UV-C system and method of the present invention, on account of combining continual long-term and short-term treatments, maintain dosage values that come as close to 4-log or greater reductions that is currently available on the market today.

The hybrid Upper Room and Whole Room UV-C disinfection system and method of the present invention functions in two separate Upper Room and Whole Room treatment modes. Upper Room treatment occurs during periods of occupancy and Whole Room treatment occurs during periods of non-occupancy. In one embodiment, the Upper Room and Whole Room operation is controlled via an app-based (iOS and Android) Bluetooth mesh control system that is integrated into the overall system of the present invention. In another embodiment, the operation is be controlled via manual wall switch for Upper Room and an app-based (iOS and Android) Bluetooth mesh control system that is integrated into the overall system of the present invention for Whole Room.

To achieve the above-mentioned purpose, the present invention provides a UV-C lighting fixture equipped with various components including a controller that is operably coupled with a user app accessible by a user through a user device. The fixture is programmed by the user to operate the fixture during occupancy and/or vacancy to maximize disinfection of interior environments in a safe manner.

In one aspect, the present invention provides a system for disinfecting an indoor environment, the system comprising: a user computer having a user app; a controller operably coupled with the user computer remotely through the user app, the user computer capable of communicating with the controller through a network; a fixture for housing the controller, the fixture further comprising: at least one UV-C lamp operably coupled with the controller, and at least one motion sensor operably coupled with the controller; wherein the at least one UV-C lamp is operable in a plurality of modes controlled remotely by a user via the user app.

In another aspect, the present invention provides a method for disinfecting an indoor environment, the method comprising the steps of: programming, by a user computer through a user app, parameters for controlling at least one UV-C lamp located remote from the user computer; communicating the parameters to a controller, through a network, the controller operably coupled with the at least one UV-C lamp and a motion sensor for detecting occupancy within the indoor environment; storing, in a memory, the parameters on the controller; and controlling, by a processor, the at least one UV-C lamp for operation in a first mode when the indoor environment is occupied, and a second mode when the indoor environment is unoccupied; wherein in the first mode, the at least one UV-C lamp is activated by the controller to emit ultraviolet radiation to an upper section of the indoor environment at a first level for occupants to safely remain within the indoor environment, and in the second mode the at least one UV-C lamp is activated by the controller to emit ultraviolet radiation to sections of the indoor environment beyond the upper section at a level greater than the first level.

In yet another aspect, the present invention provides an apparatus for disinfecting an indoor environment, the apparatus comprising: a controller operably coupled with a remote user computer having a user app, the user computer capable of communicating with the controller via a network; at least one UV-C lamp operably coupled with the controller, and at least one motion sensor operably coupled with the controller; wherein the at least one UV-C lamp is operable in a plurality of modes controlled remotely by a user via the user app.

### BRIEF DESCRIPTION OF DRAWINGS

The foregoing summary, as well as the following detailed description of presently preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a flow chart illustrating the two modes of operation of an embodiment of a system and method of the present invention;

FIG. 2 is a front perspective view of an embodiment of a fixture of the present invention with a front-facing grill of the fixture removed;

FIG. 3 is a front perspective view of the fixture of FIG. 1 in an open state;

FIG. 4 is a front perspective view of the fixture of FIG. 1 in a partially open state;

FIG. 5 is a front perspective view of the fixture of FIG. 1 in a closed state;

FIG. 6 is a front perspective view of another embodiment of a fixture of the present invention;

FIG. 7 is a bottom perspective view of the fixture of FIG. 1 in a closed state;

FIG. 8 is a wiring diagram of an embodiment of a controller of the present invention;

FIG. 9 is a flow chart illustrating the two modes of operation of another embodiment of a system and method of the present invention;

FIG. 10 is a bottom side perspective view of another embodiment of a fixture of the present invention;

FIG. 11 is a bottom perspective view of the fixture of FIG. 10 with an outer casing superimposed on the fixture;

FIG. 12 is another bottom side perspective view of the fixture of FIG. 10 with an upper lamp illuminated;

FIG. 13 is a bottom view of the fixture of FIG. 10 with the upper lamp illuminated;

FIG. 14 is a bottom view of the fixture of FIG. 10 with a lower lamp illuminated;

FIG. 15 shows mounting components for the fixture of FIG. 10;

FIG. 16 is a wiring diagram of the fixture of FIG. 10;

FIG. 17 is a top side perspective view of another embodiment of a controller of the present invention;

FIG. 18 is a wiring diagram of the controller of FIG. 17;

FIG. 19 shows an embodiment of a sensor of the present invention;

FIG. 20 shows another embodiment of a sensor of the present invention;

FIG. 21 is an illustration of a motion detection range for the sensor of FIG. 19;

FIG. 22 is illustration of a motion detection range for the sensor of FIG. 20;

FIG. 23 is an overall schematic of the system of the present invention; and

FIG. 24 is a schematic of the user app of the present invention.

To facilitate an understanding of the invention, identical reference numerals have been used, when appropriate, to designate the same or similar elements that are common to the figures. Further, unless stated otherwise, the features shown in the figures are not drawn to scale and are shown for illustrative purposes only.

### DETAILED DESCRIPTION

Certain terminology is used in the following description for convenience only and is not limiting. The article “a” is intended to include one or more items, and where only one item is intended the term “one” or similar language is used. Additionally, to assist in the description of the present invention, words such as top, bottom, side, upper, lower, front, rear, inner, outer, right and left are used to describe the accompanying figures. The terminology includes the words above specifically mentioned, derivatives thereof, and words of similar import.

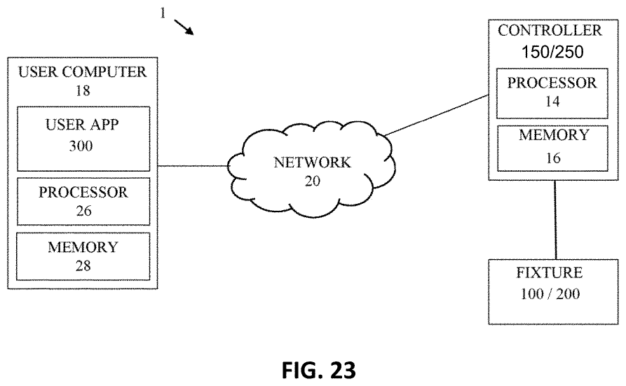

In general, as shown in FIG. 23, the system 1 of the present invention includes a fixture 100, 200 having a controller 150, 250 that communicates with a user device or computer 18 via a user app 300. The user app 300 is downloaded on a user computer 18 having a processor 26 and memory 28, with the user computer 18 connected to a network 20. The controller 150, 250, which controls various functions of the fixture 100, 200, also includes a processor 14 and memory 16. The controller 150, 250 is programmed through the user app 300 for various parameters. The network 20 could be any one of or a combination of a wireless network such as 4G, 5G, Wi-Fi or Bluetooth, or hard wired. Even though FIG. 23 is shown with only one controller 150, 250 coupled to the user app 300 via the network 20, multiple controllers could be coupled thereto such that a user is able to control multiple controllers within the same network.

### First Fixture Embodiment

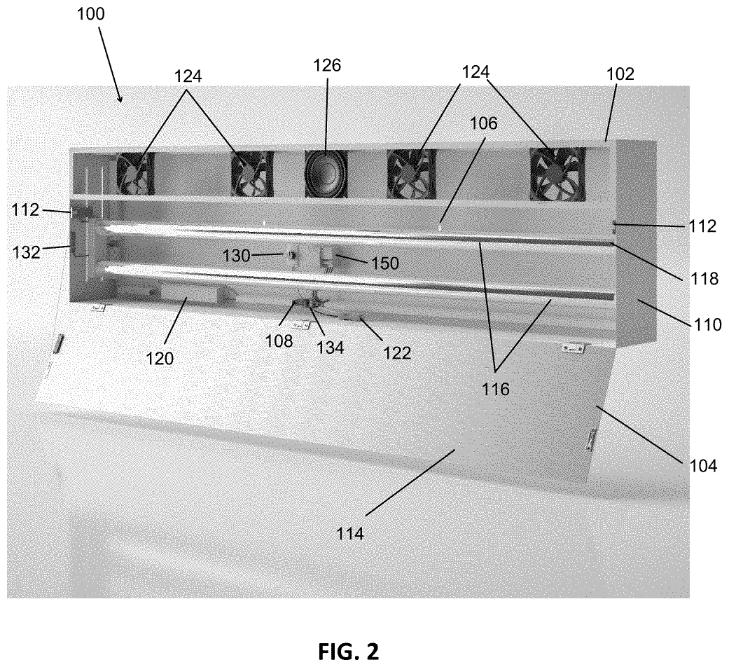

Referring to FIGS. 2-7, in one embodiment, the fixture 100 includes a housing 102 and an automated adjustable shield 104 that is pivotably coupled to the housing 102. In this embodiment, the housing 102 is constructed of 22-gauge white matte steel with a high reflectance polyester powder coat finish. Referring to FIG. 2, two pre-drilled openings 106 are provided on the housing 102 for securing the fixture 100 to a wall or ceiling. In this embodiment, each opening 106 is ¼ inch and the pair openings 106 are 16 inches apart to accommodate standard stud spacing. One of ordinary skill in the art would recognize that the openings 106 could be spaced apart at other lengths, for example, 24 inches, or further openings could be provided to accommodate for alternative mounting. For example, the housing 102 could be provided with threaded openings on a top portion for optional trunnion ceiling mount.

As well, as shown in FIG. 2, two ½ inch knockouts 108 are provided at or about the center rear of the housing 102 for power entry. The fixture 100 and its components are powered by electricity provided by a local utility, for example, 110V-277V/60 Hz AC in the U.S. However, the fixture 100 and its components could be configured to operate under other voltages and frequencies. Optionally, 120V and 277V cord and plug accessories could be provided for users who prefer not to hard-wire the fixture 100. A safety interlock switch 132 is provided to isolate power when the fixture 100 is manually opened. A power distribution block 134 is also provided for power and control wiring connections.

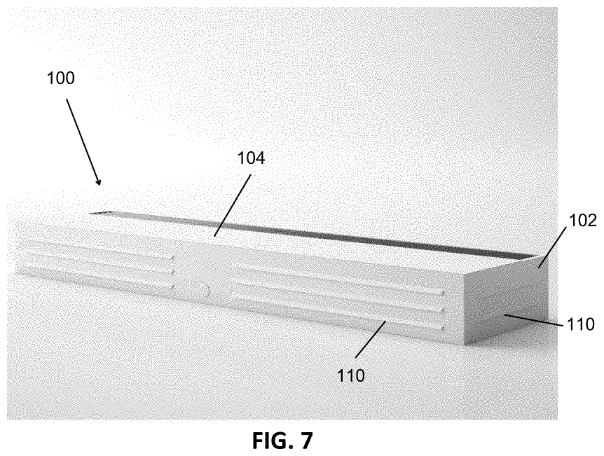

As shown in FIG. 2, two aluminum air intake grills 110 are provided on each side of the housing 102. The intake grills 110 allow for air to enter into the fixture 100. Additional intake grills 110 are provided on a bottom portion of the housing 102, as shown in FIG. 7, to further promote air flow.

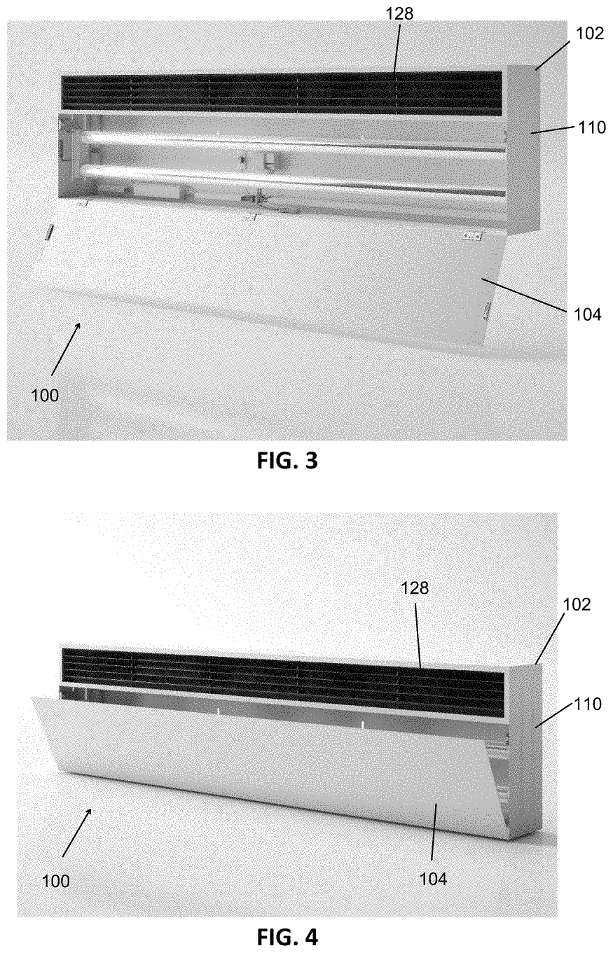

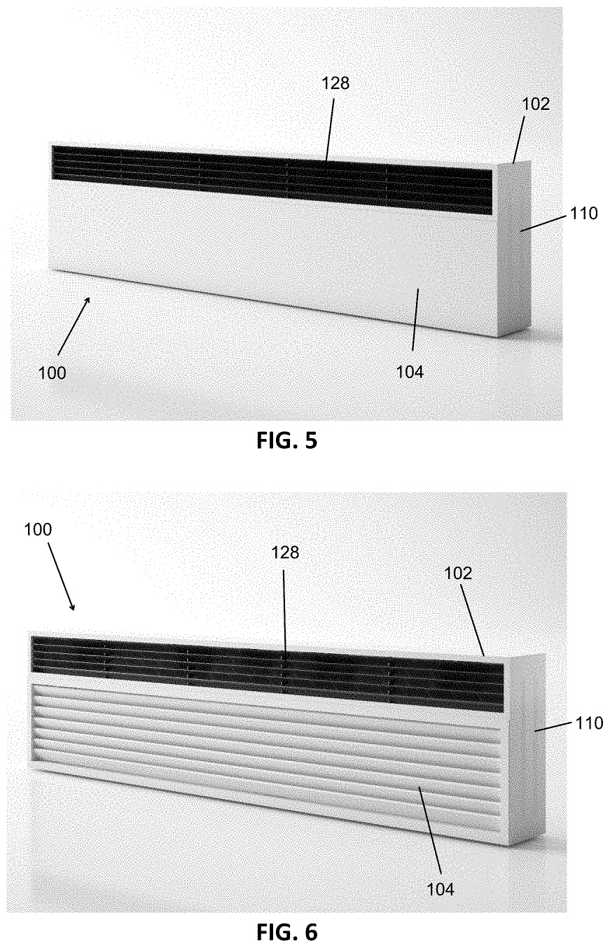

Still referring to FIG. 2, the shield 104 is operably coupled to and driven by a shield motor mechanism 112 on each side of the shield 104 such that the shield 104 is pivotable between a closed state as shown in FIGS. 5-7 when in Upper Room disinfection mode, a partially closed state as shown in FIG. 4, and an open state as shown in FIGS. 2 and 3 when in Whole Room disinfection mode. The shield 104 includes a polished specular aluminum reflector 114 on an inner surface to reflect UV-C light when in operation. The shield 104 prevents eye exposure to UV-C irradiation during the Upper Room treatment mode when the fixture 100 is in a closed state. Optionally, the shield 104 could be grated as shown in FIG. 6 to provide additional air circulation.

Still referring to FIG. 2, various components of the fixture 100 are secured to the interior of the housing 102. Two 46″ UV-C lamps 116 are mounted to UV-C resistant ceramic bi-pin lamp holders 118. In this embodiment, the UV-C lamps 116 are low pressure mercury UV-C lamps with specifications as follows—(i) lamp wattage: 37.9 W; (ii) lamp current: 0.420±0.040A; (iii) lamp voltage: 106V; (iv) tube length (L): 1,152.0±1.3 mm; (v) tube length (P-P): 1168.0 (max.); (vi) tube diameter (D): 32.5±1.55 mm; (vii) base: G13; (viii) spectral peak: 253.7 nm; (ix) UV output: 18.0 W; (x) average life: 8,000 hrs; (xi) ballast: F40T10 or G40T10; and (xii) glow starter: FG4P (JIS). The UV-C lamps 116 are operably coupled to an electronic ballast 120 by wire. In this embodiment, the ballast 120 is suitable for 80 W 2-lamp load, operational for 100-277 VAC and rated for duty cycling. As well, the electronic ballast 120 is coupled by wire to a UL-listed luminaire disconnect plug 122 to connect to input power wiring for maintenance, in compliance with the National Electrical Code (NEC). Alternatively, custom sized amalgam UV-C lamps, described below, could be used to provide longer lifespans as a result of operating at a more pressurized state.

Referring again to FIG. 2, four frictionless fans 124 and an audible alarm speaker 126 are operably mounted to an upper section of the housing 102 and covered by a front-facing grill 128, as shown in FIGS. 3-6. In this embodiment, the front-facing grill 128 is constructed of rigid plastic and painted black.

In this embodiment, the fans 124 are mounted on a narrow platform that extends horizontally from opposite sides of the fixture 100. With this configuration, a space is formed between the back portions of the fans 124 and a rear wall of the fixture 100. As a result, an unimpeded an upward air stream is formed within the fixture 100 is formed when the fans 124 are activated. Each fan 124 pulls outside air through the air intake grills 110 and pushes air out through the front-facing grill 128. The purpose of the fans 124 is twofold. First, the fans 124 pull room air in through the side vents 110 in order to clean the air and expel it through the front grill 128. Second, the fans 124 improve air mixing within the room which will improve UV-C treatment efficacy. Fan speed can be adjusted via the user app 300, which applicant provides under the trademark Intelli-Safe, which communicates with a controller 150, as will be described in more detail below.

The speaker 126 plays back pre-recorded messages during activation and operation of the fixture 100. For example, in the Whole Room treatment mode, as an added layer of protection, a non-invasive yet clearly audible message is emitted at 65 db to notify occupants to evacuate the treatment area in order to avoid being exposed to Whole Room UV-C irradiation. As well, an audible alert signals transitioning from Upper Room to Whole Room treatment modes and warns occupants to evacuate when activating Whole Room treatment.

One of ordinary skill in the art would recognize that the number and size of the fans 124 and the speaker 126 could be modified without departing from the spirit and scope of the invention.

As shown in FIG. 2, an RF motion sensor 130 is also mounted to the housing 102 and operably coupled with the controller 150. The integrated RF motion sensor 130 can detect occupants within the treatment area and maintain Upper Room treatment and/or disengage operation as per programming. In this embodiment, Whole Room treatments will only begin when the RF sensor 130 does not detect any motion for a pre-programmed time i.e. 5 minutes, but other time limits could be set as desired.

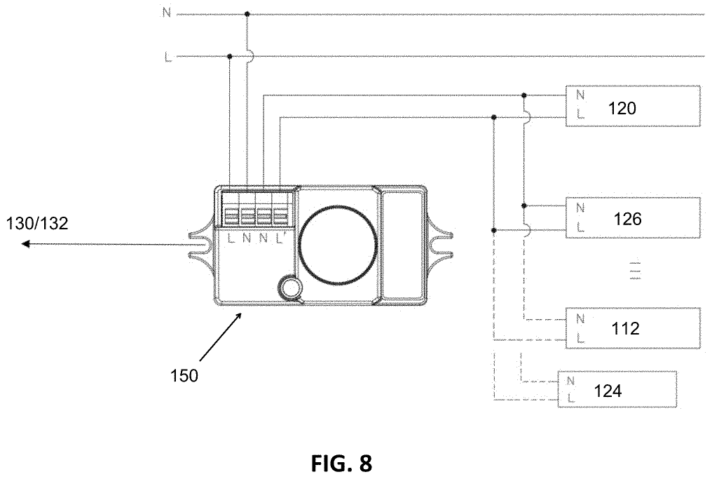

Referring to FIG. 8, the controller 150 is operably coupled to the other components of the fixture 100 by wiring with the electronic ballast 120, alarm speaker 126, shield motors 112 and fans 124 on one end, and operably coupled by wire to low voltage connections, i.e., the RF motor sensor 130 and safety interlock switch 132, on another end. The controller 150 is programmed and controlled by the user via the user app 300. In this embodiment, the user app 300 operates on iOS and Android on a mobile device. However, the user app 300 could also be used on desktop and laptop computers.

The controller 150 and the user app 300, collectively, is a Bluetooth mesh control system that controls the fixture 100 by executing pre-programmed and on-demand automated disinfection in order to ensure that Upper Room and Whole Room treatment modes are in accordance with the various features, including the exemplary features outlined above and explained in more detail below. The control system, i.e., the controller 150 and user app 300, which applicant markets under the trademark Intelli-Safe, includes many features that aid in safely disinfecting an interior environment. A remote monitoring wireless hub may be provided with the system 1 of the present invention so that the control system is connected to the user's computer network 20. As such, the user has the ability to remotely monitor status and operation of the hybrid fixtures 100.

### Operation of System with First Fixture Embodiment

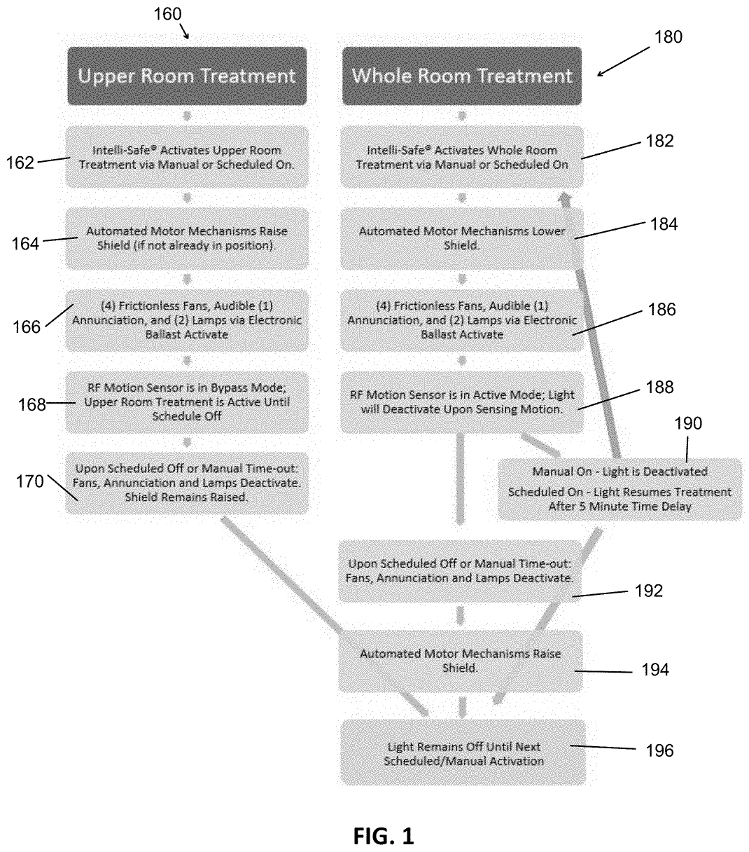

Referring to FIG. 1, in operation, once the Upper Room treatment mode 160 has been initiated via the Intelli-Safe controls (Step 162), the automated shield 104 will rise (Step 164) when switching from Whole Room mode or remain in the closed position, as shown in FIG. 5, when activating from a deactivated state. The two UV-C lamps 116 will be on along with the fans 124 (Step 166). Preferably, the fixture 100 operates at a wavelength of 254 nm but the wavelength could be varied based on utilized UV lamp technology. In addition, the speaker 126 will announce any messages that are programmed. In order to prevent any UV-C irradiation from direct eye visibility, fixtures 100 are mounted at no less than 7 ft heights from the floor in order to ensure that output threshold limits as a result of direct eye exposure are not exceeded. During Upper Room treatment, the motion sensor 130 is in bypass mode and the Upper Room treatment is active until the scheduled time to turn off (Step 168). Upon the termination of the Upper Room treatment, the lamps 116, fans 124 and speaker 126 deactivate while the shield 104 remains raised such that the fixture 100 is in a closed state (Step 170). Each of these components remain off until the next scheduled activation or manual activation (Step 196).

Still referring to FIG. 1, when Whole Room treatment 180 is initiated via the Intelli-Safe controls (Step 182) the automated shield 104 will lower (Step 184), as shown in FIG. 3, and a timed treatment will occur in accordance with the pre-programmed settings. The two UV-C lamps 116 will be on along with the fans 124 (Step 186). In addition, the speaker 126 will announce any messages that are programmed and the motion sensor 130 will be in active mode. The Hybrid UV-C fixture 100 will turn off once the Whole Room timed treatment ends in accordance with the Intelli-Safe programming. As well, if any motion is detected by the motion sensor 130 during the Whole Room treatment, then the fixture 100 will immediately deactivate for a preset period of time during which no motion is sensed (Step 188). During this situation, once the motion time-out period expires (Step 190), the fixture 100 will resume Whole Room mode (Step 182) to finish the current treatment or deactivate in accordance with the controller 150 programming. Upon the termination of the Upper Room treatment, the lamps 116, fans 124 and speaker 126 deactivate (Step 192) while the shield 104 is raised (Step 194) such that the fixture 100 is in a closed state. Each of these components remain off until the next scheduled activation or manual activation (Step 196).

### Second Fixture Embodiment

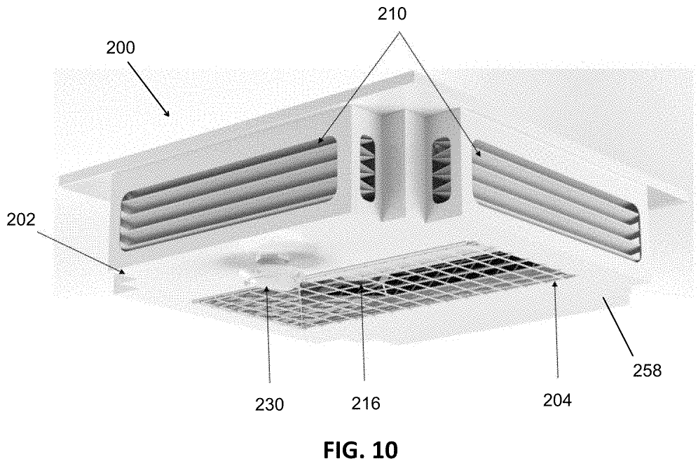

Referring to FIGS. 10-20, in another embodiment, the fixture 200 includes a housing 202 enclosed with an outer casing 258. In this embodiment, the housing 202 is constructed of 304/316 stainless steel with a polyester powder coat finish. The exterior finish is shown as white but can also be a black finish or customized color. In this embodiment, the overall dimensions of the fixture 200 are 603 mm (L)×603 mm (W)×255 mm (H). The fixture 200 and its components are powered by electricity provided by a local utility. The fixture 200 includes two (2) 300 W rated electronic ballasts 220, which are rated for 110V-277V/60 Hz AC to power corresponding 300 W UV-C lamps 216a, 216b, which are stacked against each other vertically. However, the fixture 200 and its components could be configured to operate under other voltages and frequencies. Optionally, 120V and 277V cord and plug accessories could be provided for users who prefer not to hard-wire the fixture 100.

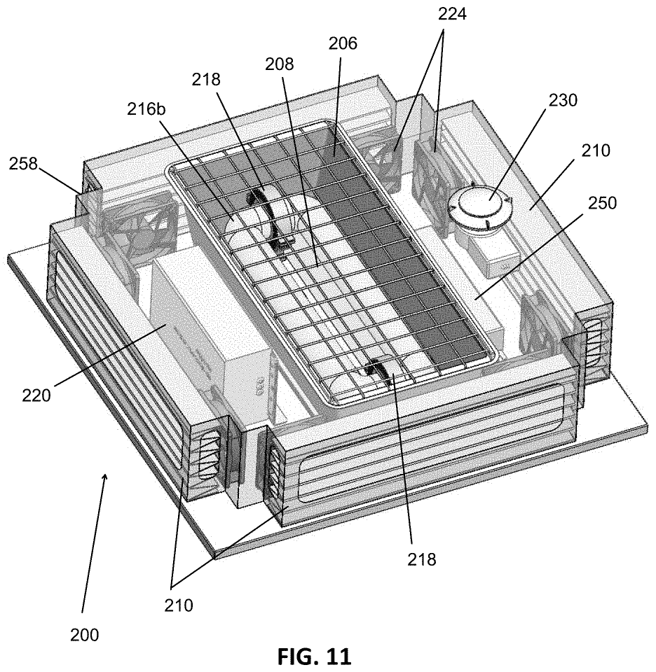

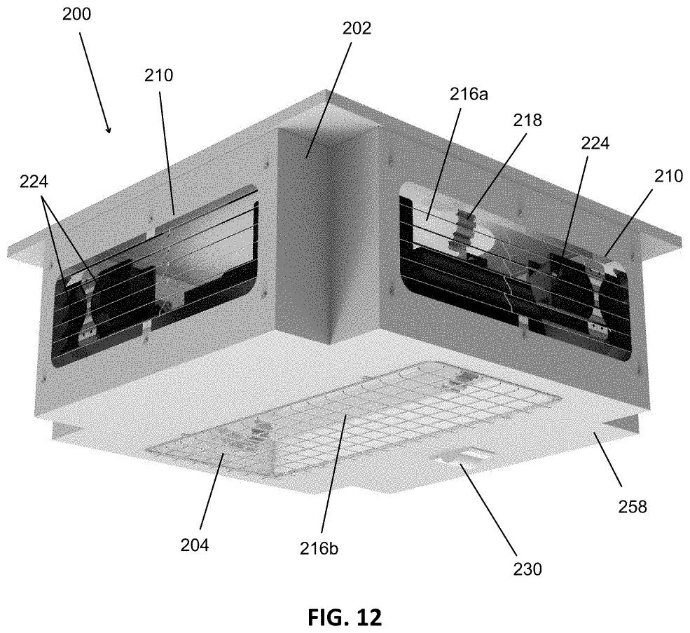

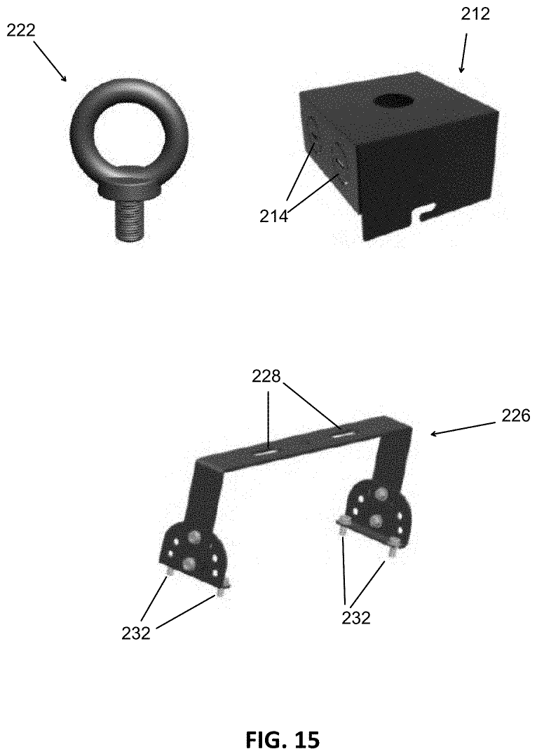

As shown in FIGS. 10-14, the fixture 200 includes a stainless-steel wire guard 204 to protect the lower UV-C lamp 216b from physical damage. A disinfection chamber 206 is provided to shield any UVC light from Upper Room operation, described in more detail below, from emitting downwards to avoid exposure. That is, a shown in FIGS. 11 and 12, the three-sided disinfection chamber 206 is formed around the lower lamp 216b to separate the upper lamp 216a and the lower lamp 216b. A power distribution block is housed on top of the fixture 200 within a 4″×4″ square junction box 212, as shown in FIG. 15, which is also provided for power and control wiring connections. The purpose of the power distribution block is to comply as a UL-listed luminaire disconnect to connect to input power wiring for maintenance, in compliance with the National Electrical Code (NEC). The junction box 212 includes four (4) ½″ knockouts 214, two (2) per opposite sides, for conduit and wire entry and is positioned in the center rear of the fixture 200.

As shown in FIGS. 10-12, four aluminum grills 210 are provided on each side of the housing 202. The grills 210 allow for UV-C light to emit from the fixture 200 for the purpose of Upper Room operation. The aluminum grills 210 are orientated at ninety degrees for the purpose of shielding any downward emitted light from direct view. For this purpose, the fixture 200 is to be mounted at heights of no less than 8 feet in order to avoid direct view of the Upper Room UV-C lamp's 216a operation during occupancy. The aluminum grills 210 are constructed of aluminum and painted black but one skilled in the art will recognize that other materials and colors could be used.

Referring to FIGS. 11 and 12, eight frictionless fans 224 are operably mounted on every corner of the housing 202. In this embodiment, the fans 224 are mounted adjacently, two per corner of the fixture 200. All eight fans 224 rotate in the same direction to expel air from the fixture 200 for the purpose of improving air mixing within the room which will improve Upper Room UV-C treatment efficacy. One of ordinary skill in the art would recognize that the number of fans 224 could be modified without departing from the spirit and scope of the invention.

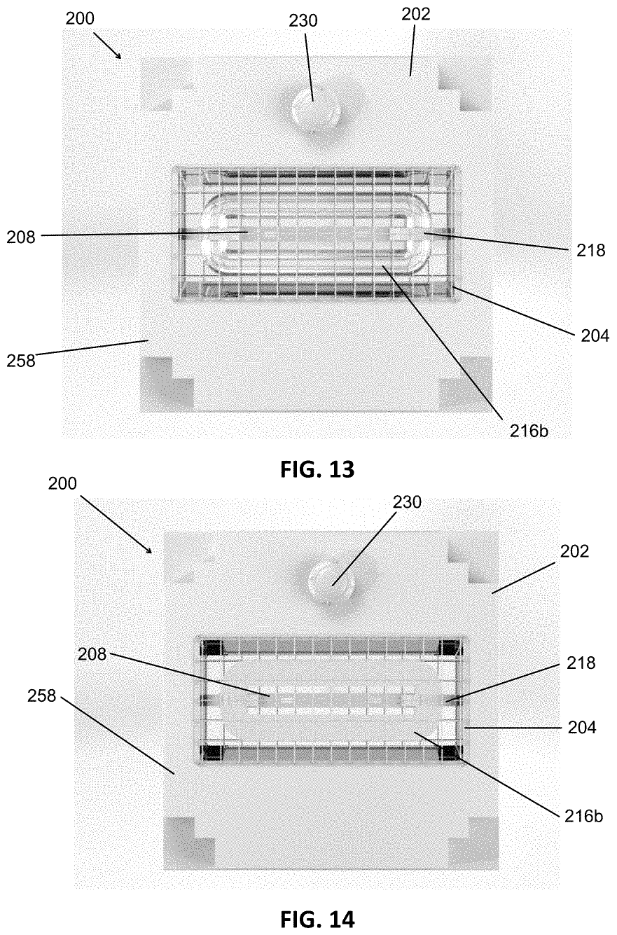

Referring to FIG. 11, various components of the fixture 200 are secured to the interior of the housing 202. Two 21.7″×6.7″ UV-C lamps 216a, 216b are mounted to four UV-C resistant ceramic lamp holders 218. As shown in FIGS. 13 and 14, the ceramic lamp holders 218 are mounted directly to a stainless-steel support bar 208, which extends between opposing ends of the lamp 216a, 216b. The lamps 216a, 216b operate at high temperature and as such, the ceramic lamp holders 218 offer thermal protection. Each ceramic lamp holder 218 features an integrated 4-pin electrical connector and wire harness for the lamps 216a, 216b to connect to. In this embodiment, the two UV-C lamps 216a, 216b are induction mercury UV-C lamps with specifications as follows—(i) size: 21.7″(L)×6.7″(W)×4″(D); power: 300 W; (iii) voltage: AC120-277V; (iv) UV strength: ≥1,300 micro watt per square centimeter at 3.5 feet; (v) effective volume: 70,500 cubic feet; (vi) UVC wavelength: 253.7 nm; (vii) lamp material: Amalgam Quartz; (viii) IP Rating: IP65; and (ix) bulb lifespan: ≥60,000 hours. Each of the UV-C lamps 216a, 216b are operably coupled to two electronic ballast 220 by wire. In this embodiment, the two ballast 220 are suitable for a 300 W 1-lamp load, operational for 100-277 VAC and rated for duty cycling. The UV-C lamps 216a, 216b and UV-C ballasts 220 are commercially available components and are not proprietary. The purpose is to allow users of fixture 200 to be able to easily source replacements parts after said components reach end of lifespan. One of ordinary skill in the art would recognize that the number and size of the lamps 216a, 216b and ballast 220 could be modified without departing from the spirit and scope of the invention.

Referring to FIG. 15, four (4) ⅜″ O-Bolts 222 are mounted in each corner on a top surface of the fixture 200 for chain mounting to a ceiling. Additionally, the O-Bolts 222 can be removed and replaced with ⅜″ threaded rods by the installer for an additional mounting method. Alternatively, the fixture 200 could be mounted with a ceiling mounting bracket 226. The ceiling mounting bracket 226 includes bolts 232 for coupling with a top surface of the fixture 200. The ceiling mounting bracket also includes two (2) oval mounting slots 228 to accept bolt sizes up to ½″. The ceiling mounting bracket 226 is for the purpose of mounting directly to the ceiling and/or adjusting the angle of tilt. The two pre-drilled openings or mounting slots 228 are ⅜″ wide and 16″ apart to accommodate standard stud spacing. One of ordinary skill in the art would recognize that these openings could be spaced apart at other lengths, for example, 24 inches, or further openings could be provided to accommodate for alternative mounting.

As shown in FIG. 11, a motion sensor 230 is also mounted to the housing 202 and operably coupled with the controller 250. Preferably, the motion sensor 230 and controller 250 are those manufactured and sold under the mark Intelli-Safe. The motion sensor 230 is a radio frequency (RF) sensor that can detect occupants within the treatment area and maintain Whole Room treatment and/or disengage operation as per programming. In this embodiment, Whole Room treatments will only begin when the motion sensor 230 does not detect any motion for a pre-programmed time i.e. 5 minutes, but other time limits could be set as desired via the user app 300. One skilled in the art would recognize that other types of motion sensors could be used as will be described below.

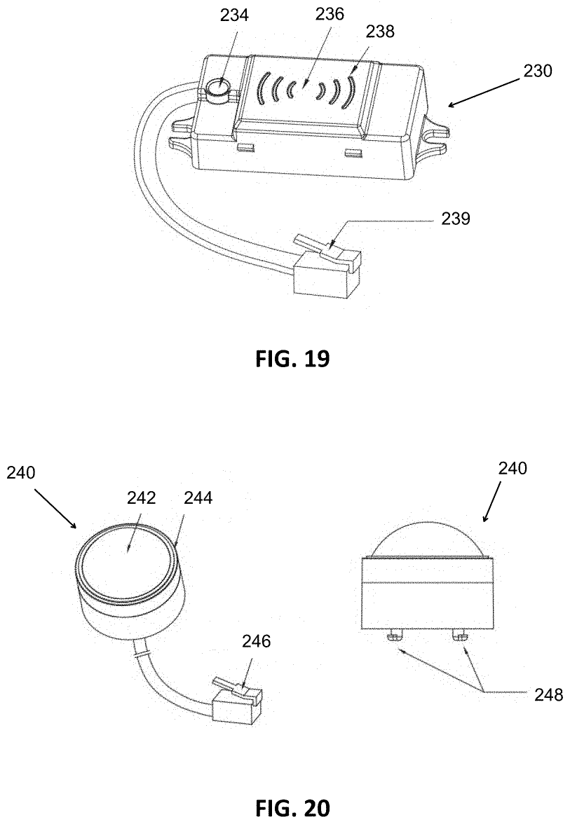

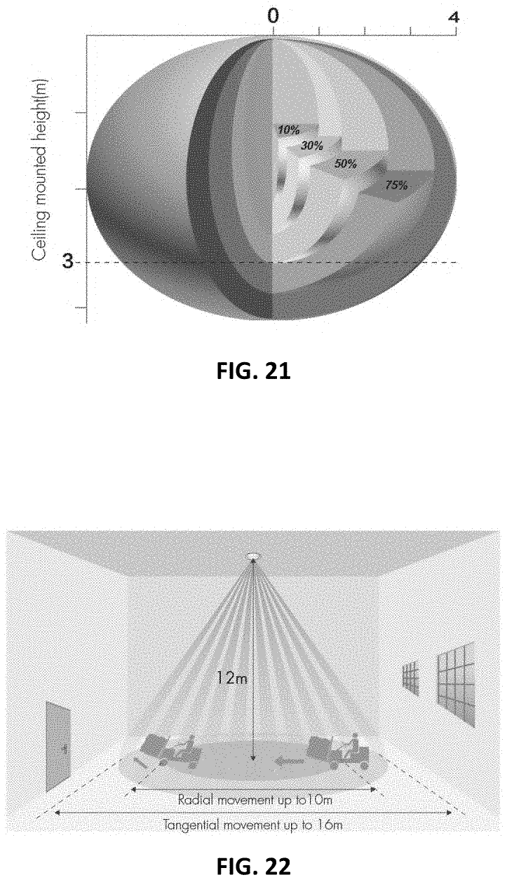

Referring to FIG. 19, the RF sensor 230 includes a daylight sensor 234, a sensor antenna 236 and a Bluetooth module 238. The RF sensor 230 is also provided with an RJ12 connector 239 for coupling with the controller 250, as shown in FIG. 18. The RF sensor 230 includes the following features—(i) sensor principle: high frequency (microwave); (ii) operation frequency: 5.8 GHz+/−75 MHz; (iii) transmission power: less than 0.2 mW; (iv) detection range: Max. (Ø×H) 8 m×3 m (as illustrated in FIG. 21); and (v) detection angle: 30 degrees-150 degrees. However, the detection range is heavily influenced by sensor placement (angle) and different walking paces. The Bluetooth module includes the following features—(i) operation frequency: 2.4 GHz-2.483 GHz; (ii) transmission power: 7 dBm; (iii) range: 10-30 m; and (iv) protocol: Bluetooth 5.0 SIG Mesh.

Referring to FIG. 20, a passive infrared (PIR) sensor 240 could also be used. The PIR sensor 240 includes a lens 242 housed within a housing 244. An RJ12 connector 246 extends from the housing 244 for coupling with the controller 250 and although not shown, the sensor 240 also includes a Bluetooth module having substantially similar features as described above with respect to the RF sensor 230. The sensor 240 includes lugs 248 on a bottom portion for mounting to the fixture housing 202. The PIR sensor 240 includes the following features—(i) sensor principle: PIR detection; (ii) operation voltage: SVDC; (iii) detection range: HIR 13× (Ø×H) 16 m×12 m; HIR 16 (L×W×H) 18 m×6 m×15 m (as illustrated in FIG. 22 based on 5 km/h movement speed); and (iv) detection angle: 360 degrees. However, the detection range is heavily influenced by sensor placement (angle) and different walking paces.

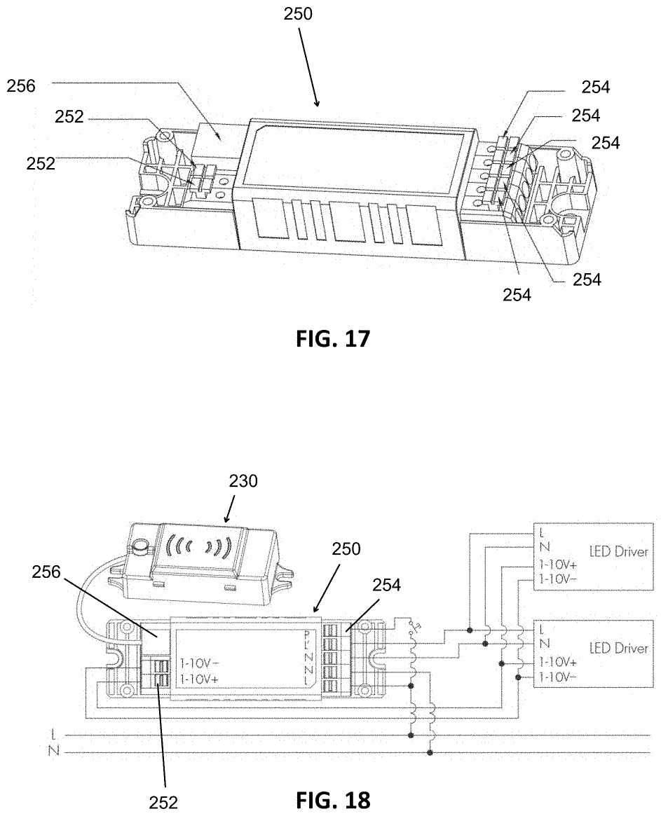

Referring to FIGS. 17 and 18, the controller 250 includes a first set of wire connectors 252 for coupling with a power supply, as well as a second set of wire connectors 254 for coupling with the various components within the fixture 200, as shown in an exemplary wiring diagram in FIG. 18. The controller 250 is also provided with a RJ12 connector 256 for coupling with the sensor 230, 240.

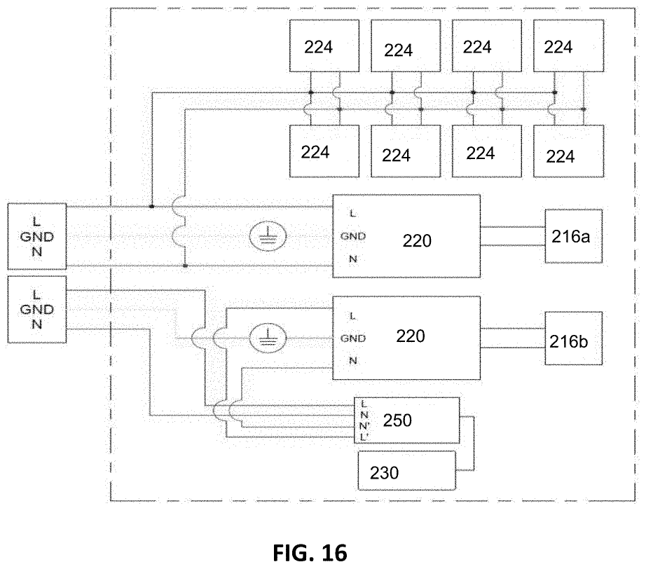

Referring to FIG. 16, the controller 250 is operably coupled by wiring with the sensor 230, one electronic ballast 220 and one UV-C lamp 216b for Whole Room operation. On the other hand, one electronic ballast 220 and one UV-C lamp 216a for Upper Room operation is connected to eight fans 224 and are operated via an external line-voltage switch, such as a light switch located in the room in which the fixture 200 is located. Input wiring connections are made within the junction box 212 at the power distribution block. The controller 250 is programmed and controlled by the user via the user app 300. In this embodiment, the user app 300 operates on iOS and Android. However, the user app 300 could also be used on desktop and laptop computers.

The controller 250 and the user app 300, collectively, is a Bluetooth mesh control system that controls the fixture 200 by executing pre-programmed and on-demand automated disinfection in order to ensure the Whole Room treatment mode is in accordance with the various features, including the exemplary features outlined above and explained in more detail below. The control system, i.e., the controller 250 and user app 300, which applicant markets under the trademark Intelli-Safe, includes many features that aid in safely disinfecting an interior environment. A remote monitoring wireless hub may be provided with the system 1 of the present invention so that the control system is connected to the user's computer network 20. As such, the user will have the ability to remotely monitor status and operation of the fixture 200.

### Operation of System with Fixture of Second Embodiment

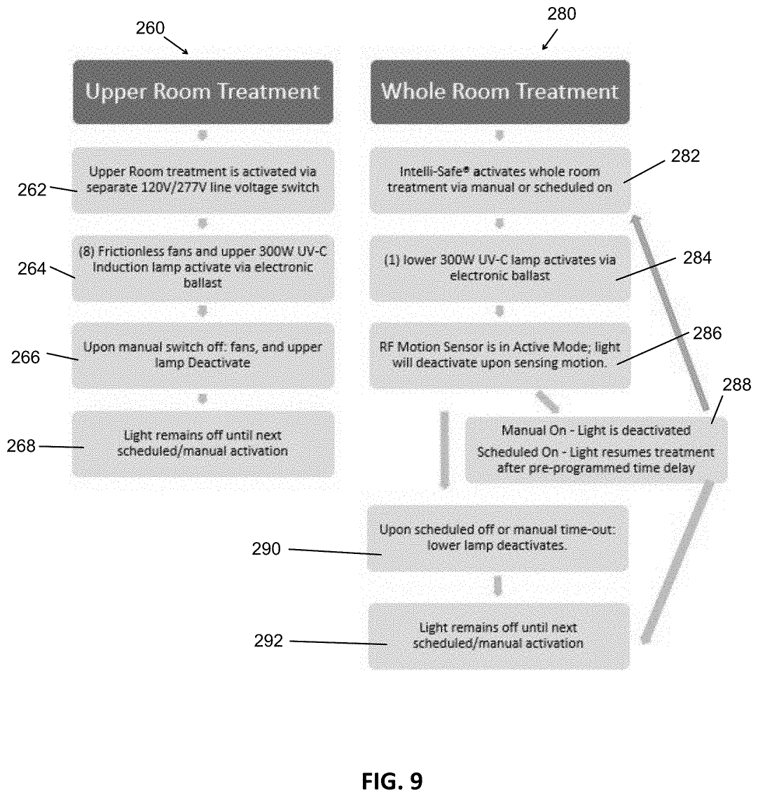

Referring to FIG. 9, in operation, once the Upper Room treatment mode 260 has been initiated via a manual switch (Step 262), eight frictionless fans 224 and one upper 300 W UV-C induction lamp 216a will activate (Step 264). Preferably, the fixture 200 operates at a wavelength of 254 nm but the wavelength could be varied based on utilized UV lamp technology. In order to prevent any UV-C irradiation from direct eye visibility, fixtures 200 are mounted at no less than 8 ft heights from the floor in order to ensure that output threshold limits as a result of direct eye exposure are not exceeded. Upon the termination of the Upper Room treatment, one Upper Room lamp 216a and eight fans 224 deactivate (Step 266) and remain off until the next manual activation (Step 268).

Still referring to FIG. 9, when Whole Room treatment 280 is initiated via the controller 250, a timed treatment will occur in accordance with the pre-programmed settings in the user app 300 (Step 282). The lower UV-C lamp 216b will be on (Step 284). The Hybrid UV-C fixture 200 will turn off once the Whole Room timed treatment ends in accordance with the controller 250 programming. As well, if any motion is detected by the motion sensor 230 during the Whole Room treatment (Step 286), then the fixture 200 will immediately deactivate for a preset period of time during which no motion is sensed (Step 288). During this situation, once the motion time-out period expires the fixture 200 will resume Whole Room mode (Step 282) to finish the current treatment or deactivate in accordance with the controller 250 programming (Step 290). Once the scheduled or manual operation time treatment completes then each of above-mentioned components remain off until the next scheduled activation or manual activation (Step 292).

### Control System and User App

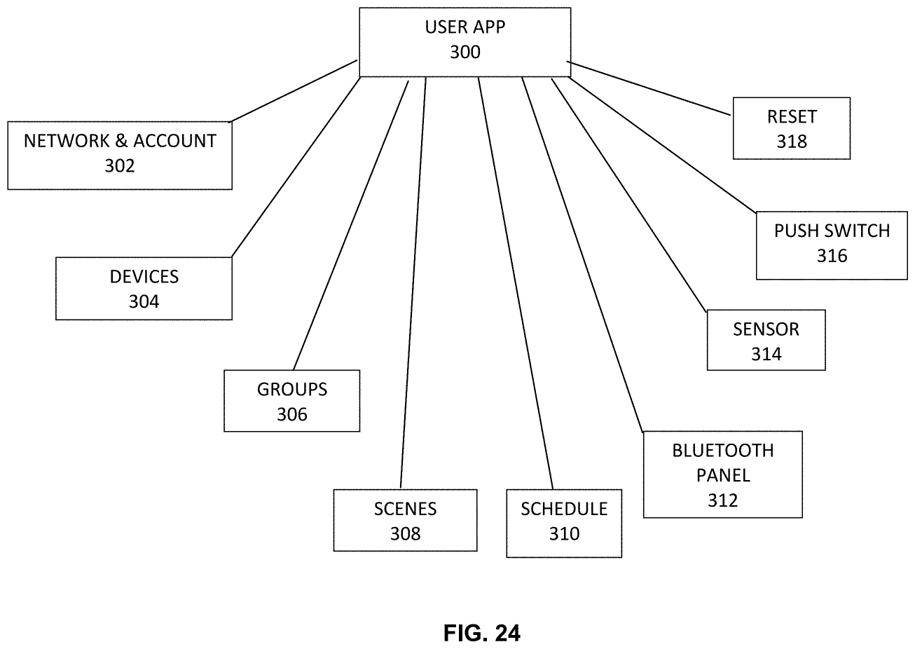

Referring to FIG. 24, numerous features and parameters are programmed in the controller 150, 250 via the user app 300 specific to the type of fixture 100, 200. The user app 300 is provided with GUIs corresponding to the numerous features and parameters for the user to select and edit. In one aspect, there are three levels of users for the user app 300: owner, installer and sub-user, which are programmed by selecting a “Network & Account” feature 302 of the user app 300. The owner is the primary user for the network and one network belongs to only one owner. The owner can control all installers and sub-accounts associated with a network. If the owner deletes the network, the network will then be removed from all the installers and sub-accounts. The owner can share the network with the installer to do commissioning work. One network can have many installers. When an installer deletes the network which is associated with multiple accounts, this same network will not be removed from other accounts, meaning that the installer cannot control other accounts associated with this network. Otherwise, the installer and owner have the same access permissions to the network. The sub-user, meaning the guest user, can use the network normally, but for some operations such as deleting the network, sharing the network to others, etc., the sub-user requires permission from the owner. Networks can be shared with a QR code or keycode, and different permission levels can be set by the network owner. The user is also able to add specific fixtures 100, 200 to manage by selecting a “Devices” feature 304 on the user app 300.

In another aspect, the user app 300 also provides different fixtures 100, 200 to be grouped through a “Groups” feature 306. Grouping ensures that multiple enabled Hybrid UV-C fixtures 100, 200 operate in accordance with the same pre-programming. Correspondingly, the grouping feature can deactivate all hybrid UV-C fixtures 100, 200 within a specific zone when only one RF sensor is activated. One luminaire or fixture 100, 200 can belong to many different groups, regardless of the other luminaries in the same group being from different rooms. For example, luminaire A in room 1 can group with luminaire B in room 2. All fixtures 100, 200 in the same group will be linked together automatically, allowing all fixtures 100, 200 within the same group to work together. As such, once a single motion sensor 130, 230 has been triggered, the others will be triggered at the same time, thus, synchronizing control.

In yet another aspect, scenes can be programmed via the user app 300 by selecting a “Scenes” feature 308. Scenes are a very useful and important function to the user. Users can create a variety of customized scenes through this feature. The scene can be recalled by the motion sensor, scheduling, push switch and/or Bluetooth panel features within the user app 300. Many types of scenes are available for users to program. In one embodiment, initially, three default scenes are available: all on, 50% on, and all off. Users can create up to 16 scenes for a single fixture 100, 200, for example, generic scene, lux on/off scene, daylight harvest scene, circadian rhythm scene and time-based scene.

The user app 300 also provides a scheduling feature, which the user could set and modify by selecting a “Schedule” feature 310. The scheduling function is another important feature for the users. With this function, the user can create a list of timers that will turn scenes on and off based on time. For example, the user can set a luminaire or fixture 100, 200 to activate during office hours, non-office hours or set corridor lights dim to a lower level at night. The user can also set a schedule based on an Astro timer (sunrise and sunset). The astronomic scheduling feature ensures that the Hybrid UV-C fixture 100, 200 operates during a pre-defined schedule and deactivates when treatments are not required. For example, a thorough long-term Whole Room treatment can be activated every night between 1 AM-2 AM for a comprehensive hands-off viral, bacterial and fungal disinfection on a routinely scheduled basis. The calendar function can be synced to the main user's IOS/Android device in order to ensure that time is accurate.

In another aspect, on-demand disinfection can be initiated by authorized users at any time via app-based controls. In one feature, the user app 300 is provided with a Bluetooth panel 312. With this feature, the user is capable of controlling the fixtures 100, 200 wirelessly. The user could also commence manual activation via the user app 300. In one embodiment, manual activation of Whole Room treatment will automatically and immediately revert to Upper-Room treatment if any motion is detected within the treatment area as an added safety measure. Alternatively, Whole Room treatment could be activated manually via the user app 300.

In another feature, the user app 300 is provided with a virtual wall switch or push switch 316. For example, the switch could activate or deactivate a particular fixture 100, 200, room or group for Upper Room or Whole Room treatment.

The user app 300 also provides settings for the UV lamps 116, 216a, 216b, which can be set and modified through the “Bluetooth Panel” feature 312. The user can change the brightness and color temperature for the luminaires or fixtures 100, 200. In this embodiment, the user app 300 provides two types of dimming: linear or logarithm. Normally, this dimming profile should be in line with the dimming pattern of Dali drivers. The user can change the load type as well. For example, it can be dimming only, or both dimming and color temperature tuning. Also, the maximum and minimum brightness and color temperature could be adjusted. Furthermore, a “status after repowered” parameter is provided to allow the user to set status of the luminaire after repowering. This is very useful for accidental power shut down. The user can choose it to remain off, stay at customized brightness and color temperature or just recover to the status before powered off. As well, method of manual mode exit could be set to program if, when and how controls will revert back to sensor control.

The user app 300 also provides settings for the sensors 130, 230, 240 of the fixtures 100, 200 through a “Sensor” feature 314. For example, the user could select a particular sensor 130, 230, 240 to control particular fixtures 100, rooms or groups, based on motion or daylight. As another example, within the sensor controls, the user is capable of further programming activations of scenes under various other parameters such as auto, semi-auto, priority and staircase function, as well as adjusting the sensitivity of the RF sensor from 10%-100% via 10% increments.

Other programmable features of the user app 300 include configuring floor plans to simplify project planning, off-line commissioning, remote control via gateway support HBGW01 and device firmware update over-the-air (OTA).

In one embodiment, the fixture 100, 200 is provided with an emergency back-up. That is, the fixture 100, 200 is programmed to deactivate in case of power loss or controller failure. The controller 150, 250 includes a built-in memory function to retain pre-programmed settings via solid state memory up to 12 weeks. The system is capable of being reset as well, through the “Reset” feature 318.

The present invention may be embodied in other specific forms without departing from its spirit or essential characteristics. The described embodiments are to be considered in all respects only as illustrative and not restrictive. The scope of the invention, therefore, will indicated by claims rather than by the foregoing description. All changes, which come within the meaning and range of equivalency of the claims, are to be embraced within their scope.

1. A system for disinfecting an indoor environment, the system comprising:
a user computer having a user app;
a controller operably coupled with the user computer remotely through the user app, the user computer capable of communicating with the controller through a network;
a fixture for housing the controller, the fixture further comprising:
at least one UV-C lamp operably coupled with the controller, and
at least one motion sensor operably coupled with the controller;
wherein the at least one UV-C lamp is operable in a plurality of modes controlled remotely by a user via the user app.

2. The system of claim 1, wherein the at least one UV-C lamp is activated by the controller in a first mode when the indoor environment is occupied, such that the at least one UV-C lamp emits ultraviolet radiation to an upper section of the indoor environment at a first level for occupants to safely remain within the indoor environment.

3. The system of claim 2, wherein the fixture further comprises at least one fan for circulating air within the indoor environment when the at least one UV-C lamp is operating in the first mode.

4. The system of claim 2, wherein the at least one UV-C lamp is activated by the controller in a second mode when the indoor environment is unoccupied, such that the at least one UV-C lamp emits ultraviolet radiation to sections of the indoor environment beyond the upper section at a level greater than the first level.

5. The system of claim 4, wherein in the second mode, the at least one UV-C lamp is inactivated by the controller when the motion sensor detects occupants within the indoor environment.

6. The system of claim 5, wherein in the second mode, the at least one UV-C lamp is re-activated by the controller when the motion sensor does not detect occupants within the indoor environment for a pre-determined amount of time.

7. The system of claim 1, wherein the at least one UV-C lamp includes an upper lamp and a lower lamp positioned below the upper lamp.

8. The system of claim 7, wherein in a first mode when the indoor environment is occupied, only the upper lamp is activated by the controller to emit ultraviolet radiation at a first level to an upper section of the indoor environment for occupants to safely remain within the indoor environment.

9. The system of claim 8, wherein in a second mode when the indoor environment is unoccupied, only the lower lamp is activated by the controller to emit ultraviolet radiation at a level greater than the first level to sections of the indoor environment beyond the upper section.

10. The system of claim 9, wherein in the second mode, the at least one UV-C lamp is inactivated by the controller when the motion sensor detects occupants within the indoor environment.

11. The system of claim 10, wherein in the second mode, the at least one UV-C lamp is re-activated by the controller when the motion sensor does not detect occupants within the indoor environment for a pre-determined amount of time.

12. A method for disinfecting an indoor environment, the method comprising the steps of:
programming, by a user computer through a user app, parameters for controlling at least one UV-C lamp located remote from the user computer;
communicating the parameters to a controller, through a network, the controller operably coupled with the at least one UV-C lamp and a motion sensor for detecting occupancy within the indoor environment;
storing, in a memory, the parameters on the controller; and
controlling, by a processor, the at least one UV-C lamp for operation in a first mode when the indoor environment is occupied, and a second mode when the indoor environment is unoccupied;
wherein in the first mode, the at least one UV-C lamp is activated by the controller to emit ultraviolet radiation to an upper section of the indoor environment at a first level for occupants to safely remain within the indoor environment, and in the second mode the at least one UV-C lamp is activated by the controller to emit ultraviolet radiation to sections of the indoor environment beyond the upper section at a level greater than the first level.

13. The method of claim 12, further comprising the step of:
inactivating, by the controller, the at least one UV-C lamp when in the second mode when the motion sensor detects occupants within the indoor environment.

14. The method of claim 13, further comprising the step of:
re-activating, by the controller, the at least one UV-C lamp when in the second mode when the motion sensor does not detect occupants within the indoor environment for a pre-determined amount of time.

15. The method of claim 14, wherein the at least one UV-C lamp includes an upper lamp and a lower lamp positioned below the upper lamp.

16. The method of claim 15, wherein in a first mode only the upper lamp is activated by the controller.

17. The method of claim 15, wherein in a second mode only the lower lamp is activated by the controller.

18. An apparatus for disinfecting an indoor environment, the apparatus comprising:
a controller operably coupled with a remote user computer having a user app, the user computer capable of communicating with the controller via a network;
at least one UV-C lamp operably coupled with the controller, and
at least one motion sensor operably coupled with the controller;
wherein the at least one UV-C lamp is operable in a plurality of modes controlled remotely by a user via the user app.

19. The apparatus of claim 18, wherein the at least one UV-C lamp is activated by the controller in a first mode when the indoor environment is occupied such that the at least one UV-C lamp emits ultraviolet radiation to an upper section of the indoor environment at a first level for occupants to safely remain within the indoor environment.

20. The system of claim 19, wherein the at least one UV-C lamp is activated by the controller in a second mode when the indoor environment is unoccupied such that the at least one UV-C lamp emits ultraviolet radiation to sections of the indoor environment beyond the upper section at a level greater than the first level.
